(19) United States
(12) Patent Application Publication
(10) Pub. No.: US 2013/0108723 A1
(43) Pub. Date: May 2, 2013

(54) Use of Plants and Plant Extracts to Activate Water, Alcohol and Other Liquids

(71) Applicant: William John Martin, South Pasadena, CA (US)

(72) Inventor: William John Martin, South Pasadena, CA (US)

(21) Appl. No.: 13/272,215

(22) Filed: Oct. 31, 2011

(51) Int. Cl. A61K 36/185; A61P 39/00

(52) U.S. Cl. 424/769

(57) ABSTRACT

The use of moringa olifera leaves and leaf powder to activate ethanol, alcoholic beverages and water is disclosed. Consumption of the energized liquids can have therapeutic benefits. The activated ethanol can be further used with neutral red dye and ultraviolet (UV) light illumination to indirectly enhance the environmental energy absorption properties of other liquids, including drinking water. Moringa activated ethanol can similarly be used with neutral red dye and UV illumination to activate the alternative cellular energy (ACE) pathway of an individual.

### CROSS REFERENCE TO RELATED APPLICATIONS

### Co-Pending Patent Application

Methods for detection of ultraviolet light reactive alternative cellular energy pigments (ACE-pigments). William John Martin Submitted Dec. 24, 2007. Publication number 20090163831

Method of assessing and of activating the alternative cellular energy (ACE) pathway in the Therapy of Diseases. William John Martin Submitted Jan. 16, 2008. Publication number 20090181467

Enerceutical mediated activation of the alternative cellular energy (ACE) pathway in the therapy of diseases. Submitted May 8, 2008. Publication number 20090280193 Regenerative wound healing using copper-silver citrate composition. Publication number: 20100099758 Submitted Oct. 22, 2008.

Enerceutical activation of the alternative cellular energy (ACE) pathway in therapy of diseases. Submitted Feb. 11, 2009. Publication number 20090202442

Method of using the body's alternative cellular energy pigments (ACE-pigments) in the therapy of diseases Submitted Feb. 20, 2009. Publication number 20100215763 Urine as a source of alternative cellular energy pigments (ACE-pigments) in the assessment and therapy of diseases. Submitted Mar. 5, 2009. Publication number 20100196297

Moringa oil mediated activation of the alternative cellular energy pathway in the therapy of diseases. Submitted Feb. 24, 2010. Publication number 20110208110

Diagnostic value of systemic ACE pathway activation in the detection by fluorescence of localized pathological lesions. Submitted Jul. 26, 2010. Publication number 20100291000

Enerceutical mediated activation of the alternative cellular energy (ACE) pathway in the therapy of diseases. Submitted July 2010.

Energy Charged Liquids to Enhance Enerceutical Activation of the Alternative Cellular Energy (ACE) Pathway in the Therapy of Diseases. Submitted Dec. 17, 2010. application Ser. No. 12/972,344

Energy Charged Alcoholic Beverages for Enhancing the Alternative Cellular Energy Pathway in the Prevention and Therapy of Diseases. Submitted January 2011. Methods for Detecting and Monitoring the Activity of Energized Water and Other Liquids Useful for Enhancing the Alternative Cellular Energy Pathway in the Prevention and Therapy of Diseases. Submitted February 2011

Methods for Increasing the Kinetic Activity of Alcohol, Water and Other Liquids, so as to Render the Liquids More Useful in Enhancing the Alternative Cellular Energy Pathway in the Prevention and Therapy of Diseases. Submitted February 2011 Methods for Increasing the Kinetic Activity of Water and Other Liquids, so as to Render the Liquids More Useful in Enhancing the Alternative Cellular Energy Pathway and in Various Other Agricultural and Industrial Applications. Submitted June 2011.

### STATEMENT REGARDING FEDERALLY SPONSORED RESEARCH OR DEVELOPMENT

Not applicable: No Federal funding was received in support of this patent application.

### REFERENCE TO SEQUENCE LISTING, A TABLE OR A COMPUTER PROGRAM LISTING COMPACT DISK APPENDIX

Not applicable.

### BACKGROUND OF THE INVENTION

It has been proposed that certain subtle properties of water can be modified by various methods, which typically utilize an external source of physical energy, such as magnets, electricity, vortex, sound and crystals. The reported beneficial properties of the treated and presumably “energized” water include amelioration of many human and animal illnesses; improved growth rate of plants; and reversal of corrosion of metal water pipes. How these effects are achieved is still controversial with a common oversimplification that the processed water is “restructured” into clusters, which differ in their sizes or in other qualities when compared to the clusters in unprocessed water. (It is usually assumed that the cluster size in the activated water is somewhat reduced or that the individual water molecules within the clusters are less tightly bound and even slightly expanded).

The issue of restructuring water is further complicated by reported observations, which imply that activated or energized water can radiate an energy field capable of improving the quality of nearby water. This approach is used by Johann Grander with a commercial device, which circulates regular or ordinary water around a sealed container of an energized water derived from an aquifer in Austria. The water flowing through Grander's device is said to be instantly changed by radiant vibration energy emanating from the water within the sealed container. Certain volcanic rocks and ceramic materials and even metal have been suggested to either naturally possess or, by using undisclosed proprietary means, can become endowed with a similar property of being able to beneficially influence the structure of water through a presumed vibration energy.

Homeopathy is yet another proposed approach to changing the properties of water. Certain therapeutic compounds are believed to achieve their beneficial effects through vibration frequencies, rather than by direct physicochemical binding interactions with receptors within the body. The vibration frequency of each particular compound is said to be unique to that compound and yet to be directly transferable to water molecules or possibly to water clusters. In producing a homeopathic formulation, a compound is first dissolved in a liquid and the solution is vigorously jolted twenty or more times in a process referred to as succussion. A small aliquot of the solution is then diluted 10 to 100 fold in water and the succussion process re-applied. Dilution of the solution followed by succession is repeated 3 to 8 times, such only a very minute amount of the original compound could be remaining. Yet the therapeutic activity of the solution is commonly considered to be enhanced at each dilution. The practice of homeopathy is further complicated by a belief that the specific frequency of vibration can somehow reverse the characteristic toxicity of the physiochemical interaction of the actual compound used to make a particular homeopathic formulation. Many of the compounds used in homeopathy are, accordingly, toxic if large amounts are ingested, producing the same symptoms for which the particular homeopathic formulation is intended to relieve. The qualitative property of specificity of each homeopathic formulation, differs from the more basic assumption that water can merely possess simply differing levels of a single quantitative energetic property.

Another aspect of water energy is revealed in studies using an electrical current to split water into hydrogen and oxygen gasses. It was initially thought by Mr. Yull Brown and other advocates of this technology that the recombining of these gases into water, as can occur when they are ignited, led to the release of substantial amounts of heat energy. Indeed, the original application of this technology was in the welding of metals. An alternative explanation for what is commonly called Brown's gas, is that electrically activated water molecules can actually assume a gaseous form, very distinct from steam, and that these gaseous molecules possess extraordinary high energy, possibly because they are somehow altered in their shape or electron carrying capacity. Beyond the use of Brown's gas for welding, gas from electrolyzed water has been stated to have healing properties when sprayed onto the skin of people with illnesses. This approach carries a potential risk since the gas is highly flammable.

Water and other liquids are also vehicles for producing and delivering a wide range of therapeutic compounds to humans. For example, colloidal silver is typically produced in water by simple electrolysis using silver electrodes. Electrolysis in sodium chloride containing water is also used to provide various anions in which the chloride ions can be shown to variously combine with oxygen atoms. In these examples, it is assumed that the active principle is the silver compound and the chloride containing anions, respectively. I would suggest, however, that these materials may instead be functioning through the modification of water. Similarly, the wound healing and plant growth enhancing quality of an electrolysis-derived copper-silver-citrate solution (patent pending), which I have described previously, may be due to the altered water, rather than to the copper and silver ions. Indeed, I have extended this line of thinking to possibly explaining how various elixirs, tinctures and other classical formulations developed by alchemists, may have achieved their proposed beneficial actions.

My interest along these lines has developed from various observations, several of which are described in the co-pending patent applications, included by reference into the present patent application. Briefly, I was able to identify an alternative cellular energy (ACE) pathway as a non-immunological cellular repair mechanism. The ACE pathway explains the reversal of the cell damaging (cytopathic) effect of viruses, which are not effectively recognized by the cellular immune system (referred to as stealth adapted viruses). Mineral containing complexes of organic molecules, termed ACE pigments, were shown to be involved in energy transduction reactions, including fluorescence, ferromagnetism, electrostasis and electron transfer. Stealth adapted virus infected patients and patients with other cellular energy deficiency diseases, including those with outbreaks of herpes simplex virus (HSV) infections and with genital warts, were shown to produce ACE pigments, which if further energized could help achieve clinical improvement in the underlying disease condition. ACE pigments could be energized using light, sound and magnetic energies. The ultraviolet light induced fluorescence of ACE pigments could be enhanced using acridine orange and neutral red dyes. The latter was of interest since it was reported that this dye could be used with UV light to expedite the healing of HSV skin lesions. Various natural compounds, referred to as enerceuticals™, were shown to possess ACE pigment-like activity and some were shown to be interactive with neutral red dye. Among the earlier enerceutical™ compositions used was a modification of a lidocaine containing mixtures of various herbs used to generate a homeopathic formulation, which I had previously confirmed as having therapeutic benefits in sick children. The modifications in the stated formulation of the homeopathic product were based on enhancing reactivity with neutral red dye. When mixed with neutral red dye and illuminated with UV light, this material was very effective in suppressing HSV skin lesions, even when a plastic barrier separated the UV illuminated solution from the actual skin lesion. This separation was also achieved by placing the solution within a plastic Ziploc bag.

Autism is one of the major illnesses caused by stealth adapted viruses. In a series of over 20 children with autism, consistent beneficial effects were observed using by placing the formulated solution plus neutral red dye over skin areas of the children, but without physical contact of the skin, and illuminating the solution with UV light. Not only was their clinical efficacy, but distinct skin areas of the children and the tongue and other areas within the mouth would also display fluorescence when UV illuminated. These studies were suspended when for then unexplained reasons, the Lidocaine containing herbal solution lost its effectiveness in achieving the desired result.

Alcohol, in the form of ethanol, was used to prepare the herbal components in the formulation and for this and other reasons, ethanol was considered important in the efficacy of the formulation. I noted and later saw that it had been previously reported that neutral red dissolved in ethanol could fluoresce. The effect was quite variable with ethanol and other alcohols. I then used various approaches suggested to be useful in activating water to see if it influenced the interaction of alcohols with neutral red. The approaches including bubbling Brown's gas (which I refer to as Water Gas) into the solution; vortexing; adding volcanic and ceramic stones and stirring with similar materials placed in a stirring wand; exposure of the solution to sound vibrations, especially around 500 hertz; succussion; and adding various minerals, including magnesium and potassium chloride. A neutral red kinetic assay (NR-Kinetic assay) was developed. It was used to demonstrate a marked increase in the kinetic movements of particles of neutral red dye sprinkled onto the processed solutions.

In these experiments, 1-2 ml of the treated ethanol solution to be tested was transferred to a small plastic container for microscopic examination. Particles of neutral red dye were then sprinkled onto the solution in the small container. Much of the particles quickly dissolved in the solution. Very fine particles, however, remained un-dissolved. Especially noticeable, even in untreated alcohol, were the linear streaks formed by the rapidly dissolving portions of the neutral red particles. Even more striking, several focused collections of residual non-dissolved material would form and from which striking to-and-fro movements of individual particles would repeatedly occur. The movement of a particle away from a cluster of particles would be sudden and much more rapid than the return of the particle back into or into the vicinity of the cluster from which it emerged. Individual particles will commonly undergo multiple to-and-fro movements. These events were particularly apparent using alcohol, which had been processed using one or more of the alcohol activation methods outlined above. Specifically, the intensity of both the formation of linear streaks by the dissolving neutral red and the to-and-fro movements of the non-dissolved residual particles was greatly enhanced by the above methods, especially when multiple methods were used on the same sample of ethanol. Moreover, the activated ethanol solutions also fluoresced more brightly with the added neutral red dye than did unprocessed ethanol. The alcohol solutions containing neutral red dye could also be filtered to remove microscopically visible non-dissolved particles and then allowed to evaporate. Interestingly, more complex and wider banding patterns of precipitating neutral red dye developed from the treated ethanol solutions when compared to the very fine banding patterns occurring with the evaporation of the filtered unprocessed ethanol plus neutral red dye solution.

The linear streaking of the dissolving neutral red dye is consistent with a directional force between regions within the solutions, which seemingly must have differing energy levels. The attraction of the non-dissolved particles into clusters and especially the very rapid repulsion of individual particles from such clusters, are also indicative of an energy force operating within the ethanol solutions. An energy force could also potentially explain the enhanced fluorescence with neutral red dye and the altered banding patterns of dissolved neutral red dye upon evaporation, which were both more clearly apparent with the treated versus the untreated ethanol.

Actual evidence for a radiating energy from the treated ethanol plus neutral red dye solutions was obtained by placing a small sealed plastic bag of the treated alcohol solutions with added neutral red dye, into a dish containing water removed from a small outdoor pond. The pond water contained numerous hydra, paramecia and other single cell microbes, which could be seen microscopically. The bag containing the alcohol plus neutral red dye was illuminated by the microscope light and also, in some experiments, by a UV lamp. Within several minutes of the bag containing the ethanol plus neutral red solution being placed into the dish, the motility of the microbes changed from clearly directional movements to purposeless circular rotations. Moreover, the organisms began to cling to one another and eventually lost their motility, showing limited or no recovery when the plastic bag containing the alcohol solution was removed from the water. This type of effect could also be achieved by holding the plastic bag containing the ethanol plus neutral red dye close to but not in direct contact with the water in which the microbes were swimming. In these studies, the activated ethanol plus neutral red dye was definitely more effective than untreated ethanol plus an equivalent amount of neutral red dye.

The ethanol and neutral red solutions were also tested for their ability to modify drinking water, as assessed using the NR-Kinetic assay. Positive effects were seen by simply floating a heat-sealed plastic bag containing an ethanol solution plus freshly added neutral red dye (generally at about 0.1 mg/ml) on water in a container, with the bag then being illuminated with a Halco 13 watt UV light bulb. Samples of the water were periodically taken for assessment using the NR-Kinetic assay. As little as several minutes and as long as several hours were employed in these preliminary studies. One hour exposure was considered adequate and convenient for most future studies, with a succussion of the plastic bag at the beginning and 30 minutes into the study.

The main purpose of the studies has been to develop simple and effective means of activating the ACE pathway in individuals in whom this pathway is likely to be deficient. It was important to find a substitute for the Lidocaine containing herbal formulation, which had not retained its activity and proved difficult to reformulate. Approximately 30 ml of coded samples of either activated or unprocessed ethanol were transferred to plastic Ziploc bags to which either neutral red dye was added or withheld, just prior to the bag being strapped onto the sole of a foot of a teenage child with autism. The bag was then illuminated with a UV light for approximately 1 hour before being removed. The child's behaviors were assessed over the next 24 hours as being either essentially unchanged or markedly improved in terms of the social interaction and communicative skills. The development or enhancement of UV fluorescence seen by UV illumination within the child's open mouth was also recorded. The results established that the activated ethanol solution with neutral red dye was markedly superior to the untreated ethanol in improving the child's performance and in enhancing intra-oral fluorescence. No clinical benefit was observed in a subsequent study in which the neutral red dye was omitted from the activated alcohol solution.

Confirmatory studies were performed in another individual, using a plastic container with ethanol plus neutral red dye, which was placed inside the mouth and illuminated with a UV light. An indirect sign of the efficacy of this approach was the appearance of direct fluorescence under UV illumination of the tongue and palate within the mouth. This change occurred within 10 minutes after initiating the treatment.

As a possible alternative approach to activating the ACE pathway, a Ziploc bag containing the activated ethanol solution with freshly added neutral red dye was tested for its capacity to energize liquids being drunk by the child with autism. With very reliable reporting, 30 minutes of prior exposure of a drinking liquids to the UV illuminated, activated alcohol with neutral red dye, achieved a clinical benefit, which had never previously been seen from simply drinking fluids. It was clearly going to be more convenient to simply have individuals regularly drink water, after it had been exposed to the fluorescing ethanol plus neutral red UV illuminated solutions.

The same approach used to transfer energy to drinking water was also used with alcoholic and non-alcoholic beverages. Essentially, the activated ethanol plus neutral red dye were sealed in a plastic bag and floated onto a container of the beverage. The bag was illuminated with UV light for varying times from 5 minutes to several hours. Samples of the water or beverage were tested using the NR-Kinetic assay and also ingested. Among the positive clinical reports from drinking the energized water and/or beverages were better sleep, more vivid dreams, more endurance and better concentration.

I have been cultivating certain medicinal plants and, in particular, moringa oleifera trees and ashitaba (Angelica keiskei) plants. The oil extracted from the leaves and seeds of moringa trees can be used directly as an enerceutical™ by applying it to the skin and illuminating it with UV light. The moringa oil is not miscible with ethanol, but the leaves and powder of moringa and ashitaba plants can be placed into alcohol. This patent application addresses the use of moringa, ashitaba and by inference other plants and plant extracts, to activate ethanol, water and other liquids to activate alcohol and other liquids as a means towards the effective and user-friendly enhancement of the ACE pathway, in individuals in whom this pathway is deficient.

### BRIEF SUMMARY OF THE INVENTION

The NR-Kinetic assay was used to demonstrate the discovery that adding freshly harvested moringa leaves or dried moringa leaf powder to ethanol or to alcoholic beverages can provide a mechanism for energizing these liquids. The energized ethanol can be used with neutral red dye for activating (energizing) other fluids, including water for drinking and for other uses. Electronic measurements of the ethanol plus neutral red dye activated water can provide supportive evidence of activation in terms of reduced electrical resistance and actual DC voltage output, detectable using electrodes placed into the water. Ashitaba and, by inference, other plants and plant extracts, can be similarly used to energize ethanol and other liquids. Among the many uses of the directly energized alcohol, or indirectly energized liquids, is the enhancement of the ACE pathway in humans, animals and plants. The cited procedures also provide a valuable tool to investigate the underlying energy acquisition and tranduction processes involved in the phenomena.

### BRIEF DESCRIPTION OF THE DRAWINGS

Not Applicable and none included

### DETAILED DESCRIPTION OF THE INVENTION

One aspect of the invention is the demonstration that moringa leaves and leaf powder are able to activate absolute ethanol as well as alcoholic beverages, as assessed by the NR-Kinetic assay. The experiments demonstrating this invention were performed as follows: Freshly harvested moringa tree leaves were chopped into small pieces, while moringa leaf powder was imported into the United Sates as a commercial product from India (Importer: Moringa Farms, Encino Calif.). For the ethanol experiments, amounts ranging from 1-10 grams of moringa leaf powder and from approximately 1-5 grams of freshly harvested moringa leaves were added to 100 ml aliquots of 200 proof absolute ethanol (Sigma-Aldrich). In the alcoholic beverages experiments, approximately 1 gram of moringa leaf powder was added to ˜20 ml of several types of alcoholic beverages, such as Ever Clear (151 proof in California), Scotch whisky (80 proof), Tequila (80 proof), red wine (27 proof), etc. At varying times, a small amount of fluid was pipetted from the test samples and placed into a small plastic container, viewable with an inverted microscope. When moringa powder was used, the kinetic movements of the fine particles of moringa powder could be directly observed and compared over time. A small quantity of neutral red dye particles was sprinkled onto aliquots from each of the treated solutions, with aliquots from untreated solutions serving as corresponding controls. The sprinkling of the neutral red powder was achieved by lightly tapping a small wooded probe (e.g. a toothpick) on which some fine neutral red dye particles had adhered; the probe being held about an inch above the solution. Once, sufficient neutral red dye had dissolved, the solution was lightly stirred and examined for its fluorescence using a Halco 13 watt, spiral UV light bulb (UV-A light at 365 nm).

Definite effects were observed within several minutes of adding the moringa powder to the absolute ethanol, alcoholic spirits and wine. In particular, the very fine particles of moringa powder showed increasing levels of kinetic activity within the various liquids, including the formation of small clusters. There were also time dependent increases in kinetic activity of the liquids, as was shown when neutral red dye particles were sprinkled onto the ethanol, alcoholic spirits and to a much lesser extent the red wine. Increasing kinetic activity was similarly observed using chopped moringa leaves, but the effects were delayed compared to those seen with moringa powder. The experiments typically lasted over several hours and at the later time points, the moringa powder and chopped leaves containing ethanol and alcoholic spirits fluoresced much more brightly with added neutral red dye, when compared to the fluorescence of the comparable control solution to which an equivalent amount of neutral red dye had been added. The intensity of fluorescence of neutral red in alcohol can be enhanced by raising the pH and this may have contributed to the finding with moringa powder and leaves. Moreover, beyond about an hour, the moringa powder treated ethanol and alcoholic spirits solutions become, themselves, directly fluorescent, yielding a red color, which is probably attributed to chlorophyll and other chemicals extracted from the moringa powder and leaves. Note that essentially identical findings with chopped moringa leaves were also obtained using chopped leaves harvested from ashitaba (Angelica keiskei) plants.

Following the activation process, the moringa powder and chopped leaves can be removed from the ethanol and alcoholic beverages by simple filtration using very fine nylon mesh and/or filter paper. The activation, measured by the NR-Kinetic assay persisted for at least several days, although it was useful to employ succussion (20-30 sharp poundings against the palm of the hand or onto a hard surface) prior to testing. The taste of the alcoholic beverages was unaltered, if not slightly improved. Although it has yet to be double blindly tested, it was felt that drinking a small amount of a moringa fortified alcoholic beverages may well have had benefits not experienced from consuming an equivalent amount of the same beverage not mied with moringa powder.

In another aspect of the invention, moringa powder was tested to see whether it could directly affect water, as assessed using the NR-kinetic assay. Moringa powder was added directly to water at room temperature. In some experiments, the water with moringa powder was heated to near boiling to prepare a moringa tea. While suggestive of some enhanced activity in the NR-Kinetic assay, the effects, if any, were not easily quantifiable. An interesting aside to this aspect of the studies was the variability seen using the NR-Kinetic assay, even with water sampled from the same container throughout the day. This observation led to the hypothesis that all water probably has an intrinsic ability to absorb radiant energies from the environment. Clearly, not all of these possible energies are understood and some may vary during the day and/or become variable for other yet unexplained reasons.

In the next aspect of the invention, an alternative method was chosen to reliably increase the NR-Kinetic activity of water and other beverages. This method was based on the previously disclosed dye/light protocol, which has been used to activate the ACE pathway in humans. Specifically, filtered moringa activated ethanol was placed into a plastic bag and sufficient neutral red dye added to provide bright UV fluorescence. As an example, approximately 0.3 mg of neutral red dye would be added to 30 ml of the activated ethanol in a 6.5″×3.25″ thin plastic bag, which was then sealed using a heat sealer. The bag was floated onto up to 1 liter of water and illuminated from above using a Halco 13 watt UV light bulb. A clearly positive effect on the NR-Kinetic assay of the water was seen within 10 minutes, although the procedure was typically allowed to proceed for several hours. The plastic bag containing the solution was occasionally succussed in some of the experiments. The NR-Kinetic enhancing activity of using UV illuminated, moringa powder activated ethanol with added neutral red dye, was confirmed as being more than that seen using untreated ethanol with added neutral red dye. Indeed, the moringa activated alcohol was essentially equivalent to that seen previously using ethanol, which had been activated using a combination of vortexing, sound and bubbling of Water Gas. (These methods are described in the most recent co-pending patent application).

In another aspect of the invention, a second assay system was developed to try to better understand the water activation process. This new system was originally based on trying to assess changes in the water's resistance to electrical conductivity using a multimeter (for example Triplett Model 9007). Not only was it found that the water resistance would progressively decline from above 100,000 ohms to below 100,000 ohms (using the 200k scale) but that occasionally actual negative values could be achieved when the 20M (Mega ohms) scale was used. More surprisingly, negative readings could also be seen using DC voltage detector on the same multimeter, when the dial was set at its lowest DC voltage reading scale of 200 mV. Inquiries to the manufacturer did not provide a meaningful explanation of what was repeatedly observed. As discussed above, it is quite possible that water can absorb and store environmental energies. Seemingly, the circuitry of the multimeter allows for easier transfer of the water energy to the positive electrode. This may possibly be based on a very minute input bias current emanating from one of the electrodes. Again, however, considerable variability was noted between voltage assays performed at different times of the day and additional work is required to fully understand the cause of this variability. What is clear, however, is the consistent increase in negative DC voltage in both water and ethanol solutions as part of the energizing process. The discovery is also consistent with the previously expressed concept that water and alcohols can apparently act as receivers of environmental energies. Moreover, especially with the addition of various compounds including neutral red dye, an energized liquid can effectively radiate a biologically active energy to other liquids.

The next aspect of the invention is the demonstration of clinical benefit from the consumption of water or other liquids, after they have been energized by contact with a UV illuminated plastic bag containing filtered, moringa activated ethanol plus neutral red dye. These studies followed earlier indications of clinical benefit, which were obtained using ethanol activated by physical means, including a combination of vortex, magnet, sound energy, plus the bubbling through the water of Water Gas (As described in the most recent co-pending patent application). The daily intake of normal quantities of water, energized by indirect exposed to UV illuminated, moringa activated ethanol plus neutral red dye, positively affected the sense of alertness, concentration and most impressively the occurrence of vivid dreaming in a test individual. The effect was noted from the first day of consuming the energized water. Other individuals tested include a young adult with a bi-polar illness and a child with autism.

In another aspect of the invention, UV illumination of a plastic bag containing filtered, moringa powder activated ethanol plus freshly added neutral red dye, was shown to be capable of activating the ACE pathway of an individual by using an earlier developed methodology. The basic procedure involved strapping the plastic bag to the sole of a foot and illuminating the bag with UV light for 60 minutes. As an alternative, a smaller plastic bag can be loosely held within the mouth, where it is UV illuminated; the subject wearing UV protective goggles. With both approaches, activation of the ACE pathway was observed both subjectively and by the development of UV fluorescence on the tongue and palate of the treated individual. Indeed, once the mouth begins to show fluorescence, the small plastic bag containing the ethanol can be removed, allowing for continued direct UV illumination on the fluorescing tongue and palate. UV illuminated moringa activated ethanol plus neutral red dye is anticipated as having benefits in many clinical conditions in which a deficiency in the ACE pathway is occurring. These conditions are specified in the co-pending patent applications and include infectious and cardio-respiratory illnesses as well as traumatic wounds.

Other aspects of the invention include the energizing of water as a means of enhancing the growth of plants. Indeed, several of the moringa trees and ashitaba plants are currently receiving water exposed to UV illuminated, moringa activated ethanol solutions. These trees and plants are being compared to those receiving ordinary water.

In a separate series of experiments, simply being noted in this application, moringa powder is being assessed for its capacity to improve mileage achievable using a gasoline fuel comprising 85% ethanol (E85). Both direct (followed by filtration) and indirect methods (the latter using a gasoline resistant container) are being evaluated. Also under evaluation are the uses of energized water and other fluids in animal husbandry, promotion of the growth and vitality of plants and in the repair of corrosion and scaling of water pipes.

In summary, a long series of original observations have helped define the ACE pathway as an important target for therapeutic interventions. Efforts have focused on simplifying the procedures capable of enhancing the ACE pathway in individuals in whom this pathway may be inadequate for retaining good health and recovering from illnesses. The present patent application records the discovery that moringa and ashitaba plant products can be used to increase the energy properties of ethanol, water and various alcoholic and non-alcoholic beverages. In all likelihood, moringa and ashitaba powder can directly activate water but the NR-Kinetic assay is not sufficiently robust to easily define an activating effect of directly added powder to the water. Instead, UV illumination of filtered, moringa powder energized ethanol with neutral red dye in a plastic bag was shown to have an indirect energizing effect on water and on other beverages. The patent application also disclosed that in addition to the previously described, NR-Kinetic assay, quantitative changes can occur in DC voltage measurements in energized water and ethanol. Claims relating to the application of electronic measurements to the study of water will be submitted in a subsequent patent application. In particular the use of advanced electronic equipment should facilitate an understanding of the source of the electrical energy being provided by the water. Indeed, energized water and alcohol may become a useful energy source beyond its potential human, animal and plant applications.

While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. A method of directly providing energy to a liquid comprising the addition to the liquid of plant material for sufficient time that if a few particles of neutral red dye are sprinkled onto the surface of an aliquot of the liquid, the particles will undergo move vigorous (kinetic) movements compared to those of a similar amount of neutral red dye sprinkled on the surface of an aliquot of the liquid obtained prior to the addition of the plant material.

2. The method of claim 1 in which the liquid is ethanol.

3. The method of claim 1 in which the liquid is an alcoholic beverage.

4. The method of claim 1 in which the liquid is water.

5. The method of claim 1 in which the liquid is a non-alcoholic beverage other than water.

6. The method of claim 1 in which the plant material comprises leaf powder or chopped freshly harvested leaves from Moringa oleifera trees.

7. The method of claim 1 in which the plant material comprises chopped freshly harvested leaves from ashitaba (Angelica keiskei) plants.

8. The method of claim 1, comprising a kit in which plant material can be automatically added to a liquid and later removed by filtration.

9. The method of claim 1 in which the liquid to which the added energy is provided is consumed as a means of enhancing an individual's alternative cellular energy (ACE) pathway.

10. A method of claim 1, comprising a kit in which plant material is retained in a liquid permeable container, which can be added to and subsequently removed from the liquid.

11. A method of indirectly providing energy to a liquid comprising placing into the liquid, a sealed plastic bag or other suitable container, in which plant material treated ethanol or alcoholic beverage is mixed with freshly added neutral red dye, and the sealed plastic bag is illuminated with an ultraviolet (UV) light, in such a manner that if a few particles of neutral red dye are sprinkled onto the surface of an aliquot of the liquid outside of the illuminated bag, the particles will undergo move vigorous movements compared to those of a similar amount of neutral red dye sprinkled on the surface of the liquid obtained prior to placement of the plastic bag and illumination with UV light.

12. The method of claim 11 in which the liquid to which the added energy is indirectly provided is consumed as a means of enhancing an individual's alternative cellular energy (ACE) pathway.

13. A method of claim 11, comprising a kit with a battery driven ultraviolet (UV) light and a compartment in which the plant material treated ethanol or alcoholic beverage can be placed with the later automated addition of neutral red dye, such that when the kit is assembled it is waterproof and can be submerged into water or other liquids to be energized.

14. A method of activating the alternative cellular energy (ACE) pathway of an individual by exposing the individual to a sealed plastic bag or other suitable container, in which plant material treated ethanol or alcoholic beverage is mixed with freshly added neutral red dye, and the sealed plastic bag is illuminated with an ultraviolet (UV) light, in such a manner that if the individual has a deficiency in the ACE pathway, areas of the body and typically including the open mouth will be seen to fluoresce if directly illuminated with a UV light and the individual will further likely report on receiving benefit from the procedure.

15. The method of claim 14 in which the sealed plastic bag is placed onto the sole of the foot or palm of the hand of the individual.

16. The method of claim 14 in which the sealed plastic bag is placed within the open mouth of the individual.

17. A method of claim 14, comprising a kit with a battery driven ultraviolet (UV) light and a compartment in which the plant material treated ethanol or alcoholic beverage can be placed with the later automated addition of neutral red dye, such that when the kit is assembled it is suitable and convenient for semi-automated application for enhancing the ACE pathway.
